(19) United States
(12) Patent Application Publication
(10) Pub. No.: US 2025/0325611 A1
(43) Pub. Date: Oct. 23, 2025

(54) IMMUNE MODULATION WITH ENHANCED ENDOTHELIAL NITRIC OXIDE SYNTHETASE ACTIVITY

(71) Applicant: 4Life Patents, LLC, Sandy, UT (US)

(72) Inventor: Xuesheng Han, Salt Lake City, UT (US)

(21) Appl. No.: 19/187,931

(22) Filed: Apr. 23, 2025

Related U.S. Application Data
(60) Provisional application No. 63/637,661, filed on Apr. 23, 2024.

(51) Int. Cl. A61K 36/13; A61K 31/519; A61K 31/714; A61P 9/00

(52) U.S. Cl. CPC A61K 36/13; A61K 31/519; A61K 31/714; A61P 9/00

(57) ABSTRACT

A nutritional supplement includes one or more immune modulators that support a subject's cellular immunity, as well as ingredients that enhance the production and use of nitric oxide by the subject's body to better regulate the subject's cellular immunity. Such a nutritional supplement may include nano-fraction immune modulators, transfer factor, or plant transfer factor, as well as sufficient amounts of vitamin B9 and vitamin B12 to increase endothelial nitric oxide synthetase activity in a body of a subject. Methods for increasing endothelial nitric oxide synthetase activity in the body of a subject are also disclosed.

### CROSS-REFERENCE TO RELATED APPLICATION

A claim for priority to the Apr. 23, 2024 filing date of U.S. Provisional Patent Application No. 63/637,661, titled IMMUNE MODULATION WITH ENHANCED ENDOTHELIAL NITRIC OXIDE SYNTHETASE ACTIVITY (“the '661 Provisional Application”). The entire disclosure of the '661 Provisional Application is hereby incorporated herein.

### TECHNICAL FIELD

This disclosure relates generally to nutritional supplements and, more specifically, to nutritional supplements that support immune health. Even more specifically, the nutritional supplements of this disclosure include immune modulators that support a subject's cellular immunity, as well as ingredients that enhance the production and use of nitric oxide by the subject's body to better regulate the subject's cellular immunity.

### SUMMARY

A nutritional supplement of this disclosure may be formulated to increase endothelial nitric oxide synthetase (eNOS) activity in a subject's body. Nitric oxide synthetases, such as eNOS, catalyze the production of nitric oxide (NO) from the amino acid L-arginine. Nitric oxide regulates cardiovascular function (e.g., by helping blood vessels relax, which improves circulation), immunity, metabolism, neurotransmission, and other biochemical processes.

In various embodiments, the nutritional supplement includes an immune modulator, vitamin B9 (folic acid, folate), and vitamin B12 (cobalamin). Optionally, the nutritional supplement may include one or more herbs and/or one or more food products (or foods) that cause and/or support enhanced eNOS activity in the body of the subject.

One or both of the vitamin B9 and the vitamin B12 may be included in the nutritional supplement in a dosage sufficient to cause a body of a subject to increase eNOS activity. The immune modulator may also be present in the nutritional supplement in a dosage sufficient to cause the body of the subject to increase eNOS activity. As an example, the vitamin B9 may be present in the nutritional supplement in an amount that will provide a dosage of at least about 1.5 mg, while the vitamin B12 may be present in the nutritional supplement in an amount that will provide a dosage of about 1 mg or more. As another example, the vitamin B9 may be present in the nutritional supplement in an amount that will provide a daily dosage of at least 2 mg, while the vitamin B12 may be present in the nutritional supplement in an amount that will provide a daily dosage of at least 4 mg.

Vitamins B6, B9, and B12 may also support healthy homocysteine levels in a body of a subject. Homocysteine an amino acid that is found in the blood when the amino acid methionine is broken down. High levels of homocysteine are indicative of damage to blood vessels. Vitamins B6, B9, and B12 may prevent damage to blood vessels, which may support the health of the heart and the cardiovascular system, help the body maintain or lower homocysteine levels, and allow the body to more efficiently produce nitric oxide.

The immune modulator may comprise any suitable immune modulator or a combination of suitable immune modulators. Without limitation, the immune modulator may comprise transfer factor (see, e.g., U.S. Pat. Nos. 4,816,563 and 6,468,534, the entire disclosures of which are hereby incorporated herein), a nano-fraction immune modulator (see, e.g., U.S. Pat. Nos. 10,471,100 and 11,197,894, the entire disclosures of which are hereby incorporated herein), a plant-based composition that modulates cellular immunity in a subject (see, e.g., U.S. patent application Ser. No. 18/922,318, the entire disclosure of which is hereby incorporated herein), or any other substance or composition that will modulate cellular immunity in a subject. In a specific embodiment, the immune modulator may comprise a combination of bovine colostrum, which includes bovine transfer factor, egg yolk, which includes avian transfer factor, and dialysate of bovine colostrum with an upper molecular weight cutoff (UMWCO) of about 5 kDa. The immune modulator may be present in the nutritional supplement in an amount that will provide a dosage sufficient to maintain eNOS activity in the body of the subject, as increased by the vitamin B9 and the vitamin B12. Optionally, the immune modulator may be present in the nutritional supplement in an amount that provides a dosage sufficient, alone or with the vitamin B9 and vitamin B12, to cause the body of the subject to increase eNOS activity. In some embodiments, one or more ingredients of the nutritional supplement (e.g., powdered colostrum, powdered egg yolk, nano-fraction of colostrum, etc.) that include the immune modulator may be included in the nutritional supplement in an amount that will provide a daily dosage of at least 100 mg. In other embodiments, the one or more ingredients of the nutritional supplement that include the immune modulator may be included in the nutritional supplement in an amount that will provide a daily dosage of at least 200 mg.

An optional herb may cause and/or support enhanced production of nitric oxide in a subject's body. Such an herb may comprise ginkgo biloba. Ginkgo biloba may be included in the nutritional supplement in an amount that will provide a daily dosage alone or with other ingredients that will scavenge nitric oxide in the body of the subject.

An optional food or food product may cause and/or support enhanced eNOS activity in the subject's body. The food or food product may be a source of nitrates, which may be used in the production of nitric oxide. Such a food or food product may comprise beet root, leafy greens (e.g., spinach, kale, chard, arugula, etc.), bok choy, etc. The food or food product may be included in the nutritional supplement in an amount that will provide a daily dosage alone or with other ingredients that will increase nitric oxide production in the body of the subject.

As another option, a nutritional supplement of this disclosure may include the amino acid L-arginine and/or the amino acid L-citrulline. eNOS produces nitric oxide from L-arginine. L-citrulline is a precursor to L-arginine.

A nutritional supplement of this disclosure may be formulated to be cardio-protective, producing a beneficial effect on the heart and/or the cardiovascular system by reducing oxidative stress and modulating inflammation responses. The nutritional supplement may facilitate a cellular defense (i.e., modulate cellular immunity) against threats to cardiovascular health, such as a high stress lifestyle, low-quality diet, and other threats to cardiovascular health.

A method for stimulating nitric oxide production by a body of a subject may include administering a nutritional supplement of this disclosure to a subject. The administration of such a nutritional supplement to the subject may support the subject's cellular immunity. The administration of such a nutritional supplement to the subject may also increase an ejection fraction of the subject's heart, which may correlate to increased production of nitric oxide by the body of the subject. An increase in the production of nitric oxide in the body of the subject may enable the subject's body to better regulate the subject's cellular immunity.

Other aspects of the disclosed subject matter, as well as features and advantages of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1A is a line graph that shows the food intake of test subjects (mice) over the sixteen (16) week study, including a control group, a group that received a high fat Paigen diet, a group that received a low dose of supplementation with a nutritional supplement of this disclosure (TFLOW), and a group that received a high dose of supplementation with the nutritional supplement (TFHIGH);

FIG. 1B is a bar graph showing the average body weights of each group of test subjects at the end of the sixteen (16) week study;

FIG. 1C is a bar graph showing the average fraction of body weights of each group of test subjects attributable to the hearts of the test subjects of each group (heart weight/body weight);

FIGS. 2A-2F are bar graphs showing average levels of total cholesterol (TC), serum triglyceride (TG), oxidized low-density lipoprotein (OX-LDL), low density lipoprotein cholesterol (LDL-C), high density lipoprotein cholesterol (HDL-C) and very low density lipoprotein (VLDL), respectively, in serum of the test subjects of each group;

FIGS. 3A-3D are bar graphs showing average levels of tumor necrosis factor-alpha (TNF-α), interleukin-1-beta (IL-1β), C-reactive protein (CRP) level, and malondialdehyde (MDA), respectively, in the serum of the test subjects of each group;

FIGS. 4A-4G are bar graphs showing average measures of ejection fraction (EF), shortening fraction (FS), heart rate, left ventricular end-diastolic anterior wall thickness (LVAW;d), left ventricular end-systolic anterior wall thickness (LVAW;s), left ventricular end-diastolic posterior wall thickness (LVPW;d), and left ventricular end-systolic posterior wall thickness (LVPW;s), respectively, of the hearts of the test subjects of each group;

FIGS. 5A and 5B are bar graphs showing average levels of lactate dehydrogenase (LDH) and creatine kinase (CK), respectively, in the serum of the test subjects of each group;

FIGS. 5C-5F are images showing markers of heart muscle damage in the hearts of the control group, the Paigen diet group, the TFLOW group, and the TFHIGH group, respectively;

FIGS. 5G and 5H are bar graphs showing average levels of alanine aminotransferase (ALT) and aspartate aminotransferase (AST), respectively, in the serum of the test subjects of each group;

FIG. 6 depicts Western blots of phosphorylated endothelial nitric oxide synthase (p-eNOS) and endothelial nitric oxide synthase (eNOS) from the hearts of the test subjects of each group, with β-actin serving as a control in the Western blots;

FIGS. 7A-7D are images showing lipid accumulation in the carotid arteries of test subjects of the control group, the Paigen diet group, the TFLOW group, and the TFHIGH group, respectively, using oil red O staining;

FIGS. 7E-7H are images showing lipid accumulation in the carotid arteries of test subjects of the control group, the Paigen diet group, the TFLOW group, and the TFHIGH group, respectively, using Masson staining; and

FIGS. 7I-7L are images showing lipid accumulation in the aortas arteries of test subjects of the control group, the Paigen diet group, the TFLOW group, and the TFHIGH group, respectively, using oil red O and hematoxylin staining.

### DETAILED DESCRIPTION

A nutritional supplement, in various embodiments, includes one or more immune modulators, as well as one or more vitamins that enhance eNOS activity in the body of a subject and, optionally, one or more herbs, foods or food products, and/or amino acids that result in increased production in nitric oxide by the body of the subject.

The one or more immune modulators may comprise any suitable immune modulator, including without limitation, transfer factor (see, e.g., U.S. Pat. Nos. 4,816,563 and 6,468,534, nano-fraction immune modulators (see, e.g., U.S. Pat. Nos. 10,471,100 and 11,197,894), plant-based substances or compositions that modulate cellular immunity in a subject (see, e.g., U.S. patent application Ser. No. 18/922,318), or the like, or combinations of any of the foregoing. The immune modulator(s) may support the immune system (e.g., by optimizing immune system function, etc.) of a subject, which may enable the subject's immune system to respond to any threat to the subject's heart and cardiovascular system.

The one or more vitamins may include vitamin B9, which may also be referred to as folic acid or folate. Vitamin B12 is believed to improve nitric oxide bioavailability in a subject's body by increasing eNOS coupling and, thus, nitric oxide production and by directly scavenging superoxide radicals. Vitamin B9 also supports the metabolism of nucleic acids and amino acids in the body and is necessary for proper red blood cell formation.

The one or more vitamins may include vitamin B12, which may also be referred to as cobalamin. Vitamin B12 is also believed to improve the bioavailability of nitric oxide in the subject's body (e.g., by enhancing eNOS activity, by scavenging nitric oxide, etc.). Vitamin B12 is believed to promote the production and maintenance of healthy levels of nitric oxide in the body. In addition, vitamin B12 supports the metabolism of carbohydrates by the body, is required for proper red blood cell formation, is essential for the formation of nerve tissue, and is essential for energy production by the body.

Optionally, the one or more vitamins may include vitamin B6, which supports macronutrient metabolism by the body and is needed for proper red blood cell formation.

Vitamins B6, B9, and B12 may also support healthy homocysteine levels in a body of a subject. Homocysteine an amino acid that is found in the blood when the amino acid methionine is broken down. High levels of homocysteine are indicative of damage to blood vessels. Vitamins B6, B9, and B12 may prevent damage to blood vessels, which may support the health of the heart and the cardiovascular system, help the body maintain or lower homocysteine levels, and allow the body to more efficiently produce nitric oxide.

Optional herbs, foods, and/or food products that are sources of nitrates may supply nitrogen that may be used by the body to produce nitric oxide. As another option, a nutritional supplement of this disclosure may include the amino acid L-arginine and/or the amino acid L-citrulline. The body uses eNOS to produce nitric oxide from L-arginine. L-citrulline is a precursor to L-arginine.

The nutritional supplement may include one or more antioxidants, which may protect the body against harmful free radicals that can harm healthy cells. Thus, the antioxidant(s) may promote healthy aging of cells in the body of a subject. In addition, the antioxidant(s) support(s) heart and cardiovascular health, the immune system, and overall wellness. The antioxidant(s) may include one or more of coenzyme Q-10 (CoQ-10), selenium (supports antioxidant activity of vitamin E; cofactor for oxidant defense enzymes), and bioactive ingredients (e.g., flavonoids, polyphenols, etc.) from Ginkgo biloba (includes flavone glycosides), garlic, red yeast rice, resveratrol, etc. CoQ-10, which is a structural component of cell membranes, is important for normal cellular metabolism, including the production of ATP (adenosine triphosphate) by mitochondria.

The nutritional supplement may also include one or more ingredients that help maintain healthy cholesterol levels in the body of a subject. For example, the nutritional supplement may include garlic and/or red yeast rice. Red yeast rice includes monacolins, which support healthy cholesterol levels.

In addition, the nutritional supplement may include magnesium.

The following tables provide specific examples of nutritional supplements that may improve and enhance regulation of a subject's cellular immunity. Each table lists the ingredients of a capsule of the nutritional supplement. A recommended daily dose for an adult is three (3) capsules (i.e., the daily dosage of each ingredient is three times (3×) the amount listed in each table) or four (4) capsules (i.e. the daily dosage of each ingredient is four times (4×) the amount listed in each table).

TABLE 1

| | Target |
|---|---|
| | Amount |
| Ingredient | (mg) |
| Vitamin B6 (Pyridoxine Hydrochloride) (HCl) | 1.39 |
| Kosher/Halal | |
| Vitamin B12 (Trit) Cyanocobalamin | 0.38 |
| Kosher only | |
| Folic Acid (Vitamin B9) | 0.23 |
| Kosher/Halal | |
| Magnesium Glycinate 18% | 19.07 |
| Kosher/Halal | |
| Magnesium (Magnesium oxide powder 58%) | 47.36 |
| Seleniummethionine (Selenium Enriched Yeast 0.2%) | 10.54 |
| CoEnzyme Q10 (from microbial fermentation) | 35.03 |
| Resveratrol (from Polygonnum cuspidatum root extract) | 6.87 |
| [50%] | |
| Ginkgo biloba leaf extract [24% Flavone | 10.00 |
| Glycosides and 6% Terpene Lactones] | |
| Red yeast rice extract [0.7% Monacolin J] | 33.33 |
| Garlic bulb extract [1% Allicin] | 100.00 |
| Dried Egg Yolk Powder (Avian) (source of transfer factor) | 10.00 |
| NanoFactor nano-fraction immune modulator | 0.67 |
| Colostrum Filtrate (source of transfer factor) | 22.67 |
| Magnesium Stearate | 8.21 |
| Silicon Dioxide | 2.05 |
| Microcrystalline Cellulose | 102.60 |

TABLE 2

| | Target |
|---|---|
| Ingredient | Amount (mg) |
| Vitamin B6 (Pyridoxine Hydrochloride) | 4.18 |
| (HCl) Kosher/Halal | |
| Vitamin B12 (Trit) Cyanocobalamin | 1.15 |
| Kosher only | |
| Folic Acid (Vitamin B9) | 0.69 |
| Kosher/Halal | |
| Vitamin B1 (Thiamine Mononitrate) | 0.59 |
| Magnesium Glycinate 18% | 28.61 |
| Kosher/Halal | |
| Magnesium (Magnesium | 71.04 |
| oxide powder 58%) | |
| CoEnzyme Q10 (from | 52.55 |
| microbial fermentation) | |
| Garlic bulb extract [1% Allicin] | 150.00 |
| Dried Egg Yolk Powder (Avian) | 15.00 |
| NanoFactor | 1.00 |
| Colostrum Filtrate | 34.00 |
| Magnesium Stearate | 9.80 |
| Silicon Dioxide | 2.50 |
| Microcrystalline Cellulose | 125.49 |

TABLE 3

| | Target |
|---|---|
| Ingredient | Amount (mg) |
| Vitamin B6 (Pyridoxine Hydrochloride) (HCl) | 4.18 |
| Kosher/Halal | |
| Vitamin B12 (Trit) Cyanocobalamin | 1.15 |
| Kosher only | |
| Folic Acid (Vitamin B9) | 0.69 |
| Kosher/Halal | |
| Magnesium (Magnesium oxide powder 58%) | 79.91 |
| Seleniummethionine (Selenium Enriched Yeast 0.2%) | 15.81 |
| CoEnzyme Q10 (from microbial fermentation) | 52.55 |
| Red yeast rice extract [0.7% Monacolin J] | 50.00 |
| Dried Egg Yolk Powder (Avian) | 15.00 |
| NanoFactor | 1.00 |
| Colostrum Filtrate | 34.00 |
| Microcrystalline Cellulose | 87.69 |
| Magnesium Stearate | 7.02 |
| Silicon Dioxide | 1.75 |

TABLE 4

| | Target |
|---|---|
| | Amount |
| Ingredient | (mg) |
| CoEnzyme Q10 (from microbial fermentation) | 52.55 |
| Resveratrol (from Polygonnum cuspidatum root extract) | 10.30 |
| [50%] | |
| Ginkgo biloba leaf extract [24% Flavone Glycosides | 15.00 |
| and 6% Terpene Lactones] | |
| Red yeast rice extract [0.7% Monacolin J] | 50.00 |
| Garlic bulb extract [1% Allicin] | 150.00 |
| Dried Egg Yolk Powder (Avian) | 15.00 |
| NanoFactor | 1.00 |
| Colostrum Filtrate | 34.00 |
| Silicon Dioxide | 1.65 |

The next table provides another specific example of a nutritional supplement that may improve and enhance regulation of a subject's cellular immunity. The table lists the ingredients of a capsule of the nutritional supplement. A recommended daily dose for an adult is three (4) capsules (i.e. the daily dosage of each ingredient is three times (3×) the amount listed in the table).

TABLE 5

| | Target |
|---|---|
| Ingredient | Amount (mg) |
| Vitamin B6 (Pyridoxine Hydrochloride) (HCl) | 0.98 |
| Kosher/Halal | |
| Vitamin B12 (Trit) Cyanocobalamin | 0.00333 |
| Kosher only | |
| Folic Acid (Vitamin B9) | 0.166 |
| Kosher/Halal | |
| Magnesium Glycinate 18% | 3.33 |
| Kosher/Halal | |
| Magnesium (Magnesium oxide powder 58%) | 26.7 |
| Seleniummethionine (Selenium Enriched Yeast 0.2%) | 0.020 |
| CoEnzyme Q10 (from microbial fermentation) | 33.3 |
| Resveratrol (from Polygonum cuspidatum root | 3.33 |
| extract-50%) | |
| Ginkgo biloba leaf extract (24% Flavone | 10 |
| Glycosides and 6% Terpene Lactones) | |
| Red yeast rice extract [0.7% Monacolin J] | 33.3 |
| Garlic bulb extractd (1% Allicin) | 100 |
| Tri-Factor | 33.3 |

A study was conducted to evaluation the protective effects of the embodiment of the nutritional supplement of TABLE 5 (“TF Cardio”) on the heart and cardiovascular health of mice. The procedure and results of that study follow.

Five-week-old C57BL/6J female mice were purchased from Gempharmatech Co. After one week of adaptive feeding, the mice were divided into four groups, namely, a control group (n=15), which was given control chow plus water by gavage; a model group (“Paigen group,” n=15), which was given a high-fat diet (15% fat 1.25% cholesterol, and 0.5% choline salt) plus water by gavage; a low-dose intervention group (“TFLOW group,” n=15), which was given the high-fat diet plus a low dosage of TF Cardio (152.5 mg/kg) by gavage; and high-dose intervention group (“TFHIGH group,” n=15), which was given the high-fat diet plus a high dosage of TF Cardio (762.5 mg/kg) by gavage. The mice were fed/treated in this manner for 16 weeks.

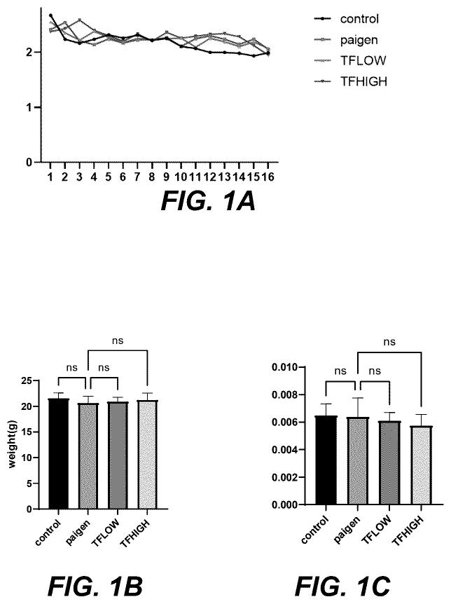

As shown in FIGS. 1A-1C, none of the Paigen diet, the low dose of TF Cardio, or the high dose of TF Cardio affected the food intake of the mice (FIG. 1A), the body weight of the mice (FIG. 1B), or altered the heart weight of mice relative to the body weight of the mice (FIG. 1C).

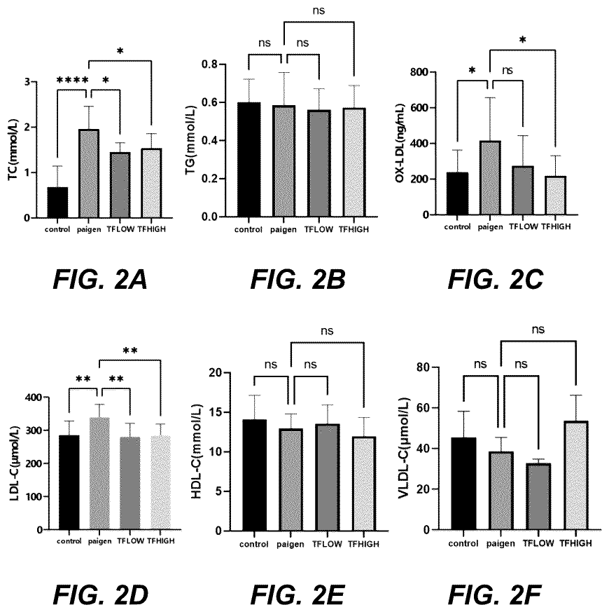

As shown in FIGS. 2A, 2C, and 2D, the Paigen diet induced an increase in the total cholesterol (TC) (FIG. 2A), oxidized low-density lipoprotein (OX-LDL) (FIG. 2C), and low density lipoprotein cholesterol (LDL-C) (FIG. 2D) levels in the serum (blood) of mice. As shown in FIGS. 2A, 2C, and 2D, a high dose of TF Cardio reduced the paigen diet-induced elevation of serum TC (FIG. 2A), OX-LDL (FIG. 2C), and LDL-C (FIG. 2D), while a low dose of TF Cardio decreased the level of paigen diet-induced elevation of serum TC (FIG. 2A) and LDL-C (FIG. 2C). As further shown by FIG. 2D, the cholesterol-lowering effect of TF Cardio did not show a dose-dependent response. As shown by FIGS. 2B, 2E, and 2F, no statistically significant differences (ns) were found between serum triglyceride (TG) (FIG. 2B), high density lipoprotein cholesterol (HDL-C) (FIG. 2E), or very low density lipoprotein (VLDL-C) (FIG. 2F) in the four groups of mice.

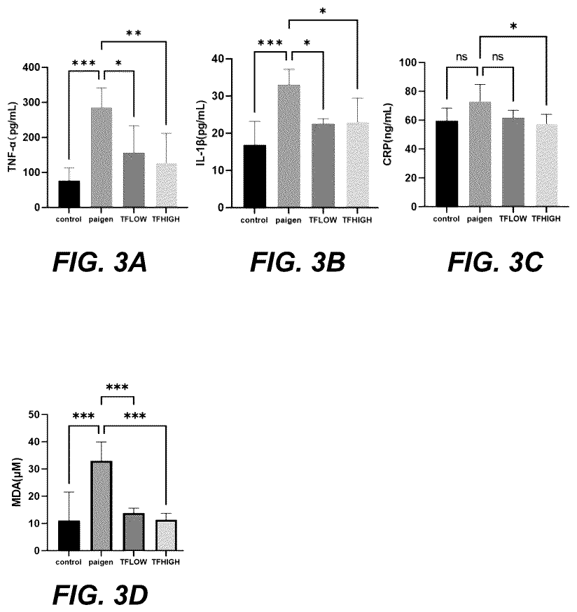

FIGS. 3A-3C show the levels of TNF-α, IL-1β, and CRP in the serum of mice from the control group, the Paigen group, the TFLOW group, and the TFHIGH group. Serum was collected and tested using an ELISA kit with an absorbance measurement obtained at 450 nm. As depicted by FIGS. 3A and 3B, the Paigen diet the Paigen diet induced an increase in serum TNF-α (FIG. 3A) and IL-1β (FIG. 3B) levels in mice, while both a low dose of TF Cardio and a high dose of TF Cardio reduced the levels of serum TNF-α (FIG. 3A) and IL-1β (FIG. 3B). As shown in FIG. 3C, the Paigen diet did not result in a statistically significant (ns) increase in the CRP level in the serum, but there was a statistically significant (*) decrease in the CRP level in mice treated with a high dose of TF Cardio. TNF-α, IL-1β, and CRP are markers of inflammation. Thus, while the Paigen diet increased inflammation in mice, the TF Cardio reduced inflammation.

FIG. 3D shows the effects of the Paigen diet, low dose TF Cardio treatment, and high dose TF Cardio treatment on levels of MDA in the serum. MDA is an important index of oxidative stress. The Paigen diet induced an increase in the level of MDA in the serum of mice and, thus, increased oxidative stress in the mice. Both the low dose TF Cardio treatment and the high dose of TF Cardio treatment decreased the level of MDA in the serum of mice, reducing oxidative stress in the mice.

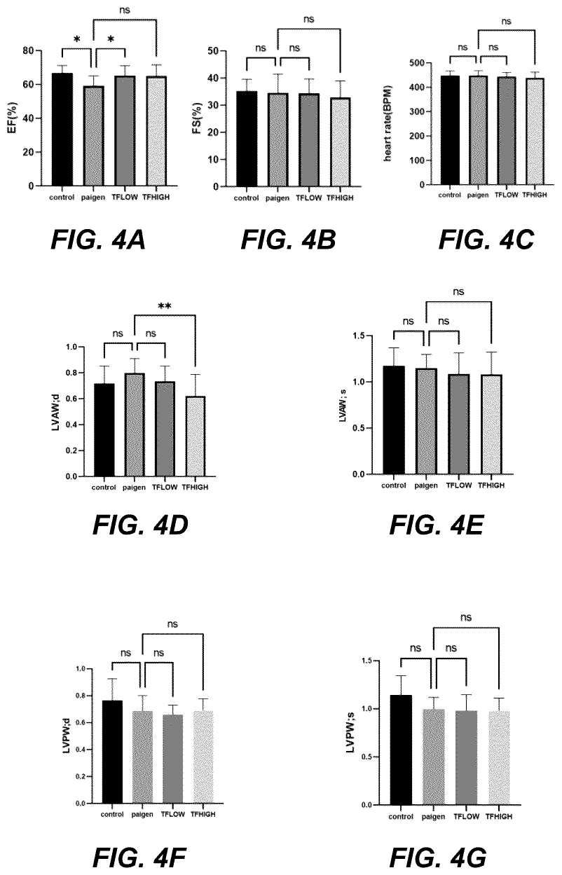

After sixteen (16) weeks of feeding, transthoracic echocardiography was performed on all mice. The mice were fixed on a thermostatic heating plate at 37° C. under 1.5% isoflurane inhalation. The hair was removed on the chest and upper abdomen to fully expose the skin around the heart, and echocardiography was performed at a heart rate of about 450 beats per minute (BPM (using an MS-550D probe of a Vevo® 3100 small animal ultrasound machine was applied for detection. A left ventricular long-axis section was taken and the M mode and B mode data of the mice were recorded. Based on the left ventricular long-axis section, the probe was rotated 90° clockwise, which was the left ventricular short-axis section, and the M mode and B mode data of the mice were recorded. Ejection fraction (EF) (FIG. 4A), shortening fraction (FS) (FIG. 4B), heart rate (FIG. 4C), left ventricular end-diastolic anterior wall thickness (LVAW;d) (FIG. 4D), left ventricular end-systolic anterior wall thickness (LVAW;s) (FIG. 4E), left ventricular end-diastolic posterior wall thickness (LVPW;d) (FIG. 4F), and left ventricular end-systolic posterior wall thickness (LVPW;s) (FIG. 4G) were measured. All of the above ultrasound measurements were averaged over three (3) consecutive cardiac cycles.

As the graph of FIG. 4A indicates, echocardiography showed that ejection fraction (EF) decreased in the mice of the Paigen group and that both low dose TF Cardio and high-dose TF Cardio improved the EF (i.e., the increase does not appear to be dose-dependent). Echocardiography also showed that, over the course of the study, none of the Paigen diet, low dose TF Cardio treatment, or high dose TF Cardio treatment had any statistically significant effect (ns) on shortening fraction (FS) (FIG. 4B), left ventricular end-systolic anterior wall thickness (LVAW;s) (FIG. 4E), left ventricular end-diastolic posterior wall thickness (LVPW;d) (FIG. 4F), or left ventricular end-systolic posterior wall thickness (LVPW;s) (FIG. 4G).

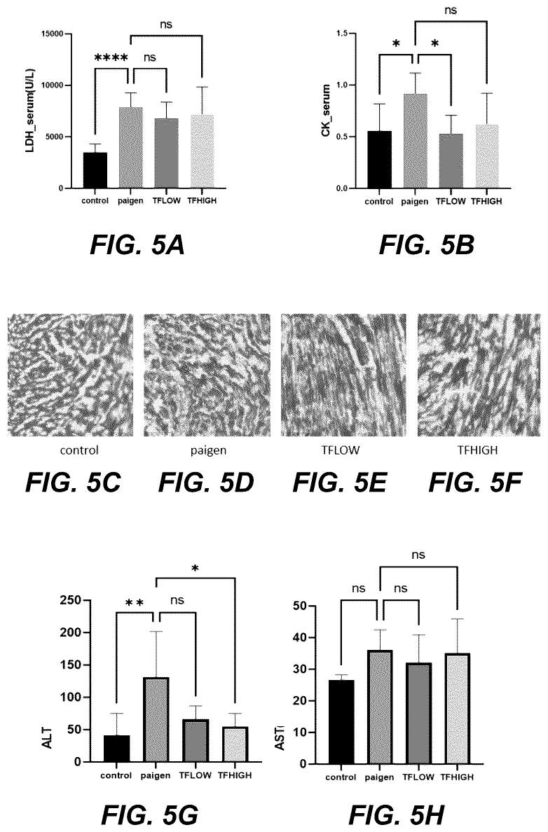

FIGS. 5A, 5B, 5G, and 5H show that TF Cardio may protect against damage to the heart (FIGS. 5A and 5B) and liver (FIGS. 5G and 5H). More specifically, as shown by the graphs of FIGS. 5A and 5B, respectively, levels of lactate dehydrogenase (LDH) and creatine kinase (CK) in the serum of the mice of the control group, the Paigen group, the TFLOW group, and the TF high group were determined using an ELISA kit with an absorbance measurement obtained at 450 nm. FIGS. 5A and 5B respectively show that while the Paigen diet induced elevation in LDH and CK levels, added supplementation with a low-dose of TF Cardio could reduce the increase in CK levels (FIG. 5B).

FIGS. 5G and 5H show that TF Cardio may protect against liver damage. Alanine aminotransferase(ALT) (FIG. 5G) and aspartate aminotransferase (AST) (FIG. 5H) are indexes of liver damage. As shown in FIG. 5G, the Paigen diet induced an increase in ALT levels in the Paigen group, while ALT levels were much lower in the TFHIGH group (p<0.05). As shown in FIG. 5H, no statistically significant (ns) difference was seen in the levels of AST among the four groups (p>0.05).

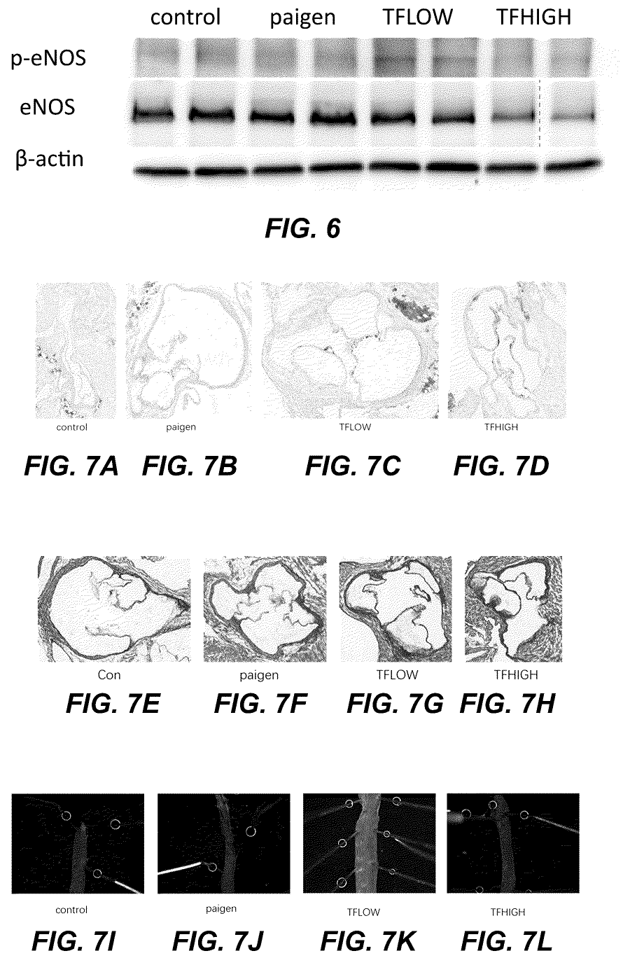

Turning now to FIG. 6, eNOS and p-eNOS were detected by Western blot analysis. The expression of both eNOS and p-eNOS decreased from the control group to the Paigen group, while the expression of p-eNOS increased significantly from the Paigen group to the TFLOW group.

After the intervention period, the mice in each group were fasted overnight and anaesthetized. Blood was drawn from the canthus and serum was collected. The mice were then sacrificed. The heart was exposed and slowly perfused with 10 mL of phosphate buffered saline (PBS). After perfusion, the carotid arteries, heart, aorta, and liver were collected. The bottom tip of the heart was put into the optimal cutting temperature compound, and 8 μm thick frozen sections were cut and stored in the freezer at −80° C.

Frozen sections of aortic sinus were rewarmed at 26° C. for 15 minutes, fixed in 4% paraformaldehyde for 15 minutes, and soaked in 60% isopropyl alcohol for 10 seconds. The sections were placed in oil red O working solution in the dark for 15 minutes, and then washed with 60% isopropanol. Then the sections were stained with hematoxylin staining solution for 20 seconds. The sections were then sealed with glycerol gelatin.

Heart tissue was fixed in 4% paraformaldehyde and embedded in paraffin. Five micron (5 μm) thick slices were cut for staining with Masson's trichrome stain. The level of myocardial fibrosis was observed by Masson's trichrome staining.

FIGS. 5C, 5D, 5E, and 5F respectively show tissue samples stained with Masson's trichome stain from the control group, the Paigen group, the TFLOW group, and the TFHIGH group. While some myocardial fibrosis was seen in the control group (FIG. 5C) and significantly more myocardial fibrosis was seen in the Paigen group (FIG. 5D), very little, if any, myocardial fibrosis was seen in the TFLOW group (FIG. 5E) or the TFHIGH group (FIG. 5F). While TF Cardio is believed to prevent myocardial in subjects who consume high fat diets or even reduce myocardial fibrosis, there does not appear to be dose-dependent relationship between the TF Cardio and the amelioration or reduction in myocardial fibrosis.

The results of the oil red O staining, which was used to evaluate the effect of vascular lipid deposition in the mice, are shown in FIGS. 7A-7D, with FIG. 7A showing a carotid artery sample from the control group, FIG. 7B showing a carotid artery sample from the Paigen group, FIG. 7C showing a carotid artery sample from the TFLOW group, and FIG. 7D showing a carotid artery sample from the TFHIGH group.

Carotid artery samples that were stained with Masson's trichrome stain are shown in FIGS. 7E-7H, with FIG. 7E showing a carotid artery sample from the control group, FIG. 7F showing a carotid artery sample from the Paigen group, FIG. 7G showing a carotid artery sample from the TFLOW group, and FIG. 7H showing a carotid artery sample from the TFHIGH group.

Aorta samples that were stained with oil red O and hematoxylin are shown in FIGS. 7I-7L, with FIG. 7I showing a carotid artery sample from the control group, FIG. 7J showing a carotid artery sample from the Paigen group, FIG. 7K showing a carotid artery sample from the TFLOW group, and FIG. 7L showing a carotid artery sample from the TFHIGH group.

The vasculature of mice in this model did not show significant lipid deposition in either the carotid or the aortic artery.

Feeding C57BL/6 mice with the Paigen diet for sixteen (16) weeks induces dyslipidemia, inflammation, heart, and liver damage, as will as mild heart dysfunction. A composition of this disclosure (e.g., TF Cardio, etc.) has a protective effect against dyslipidemia, inflammation, and damage to the heart and liver. A composition of this disclosure also has some protective effect against heart dysfunction (e.g., against decreased ejection fraction (EF), etc.), indicating that a composition of this disclosure may activate eNOS in the heart and elsewhere in the body, which may increase levels of nitric oxide (NO) in the body.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

What is claimed:

1. A nutritional supplement, comprising:
an immune modulator; and
vitamin B9 and B12, at least one of the vitamin B9 and the vitamin B12 included at a dosage sufficient to cause a body of a subject to increase endothelial nitric oxide synthetase activity.

2. The nutritional supplement of claim 1, wherein the immune modulator and the vitamin B9 and vitamin B12 are included at dosages sufficient to cause the body of the subject to cause the body of the subject to increase the endothelial nitric oxide synthetase activity.

3. The nutritional supplement of claim 2, wherein:
the vitamin B9 is present at a dosage of at least about 1.5 mg; and
the vitamin B12 is present at a dosage of about 1 mg or more.

4. The nutritional supplement of claim 2, wherein:
the vitamin B9 is present at a dosage of at least 2 mg; and
the vitamin B12 is present at a dosage of at least 4 mg.

5. The nutritional supplement of claim 2, wherein the immune modulator is present at a dosage of at least 100 mg.

6. The nutritional supplement of claim 2, wherein the immune modulator is present at a dosage of at least 200 mg.

7. The nutritional supplement of claim 1, wherein the immune modulator comprises transfer factor and/or a nano-fraction immune modulator.

8. The nutritional supplement of claim 1, wherein the immune modulator is a plant-based immune modulator.

9. The nutritional supplement of claim 1, further comprising:
an herb in a dosage that causes the body of the subject to increase the endothelial nitric oxide synthetase activity.

10. The nutritional supplement of claim 9, wherein the herb comprises ginkgo biloba.

11. The nutritional supplement of claim 1, further comprising:
a food product in a dosage that causes the body of the subject to increase nitric oxide production.

12. A method for stimulating nitric oxide production by a body of a subject, comprising:
administering a composition including an immune modulator, vitamin B9, and vitamin B12 at a dosage in which an amount the vitamin B9 and an amount of the vitamin B12 are sufficient to increase endothelial nitric oxide synthetase activity in the body of the subject.

13. The method of claim 12, wherein administering the composition comprises administering the composition with the immune modulator maintaining or further increasing the endothelial nitric oxide synthetase activity in the body of the subject, as increased by the vitamin B9 and the vitamin B12.

14. The method of claim 12, wherein administering the composition increases an ejection fraction of the heart of the subject.

15. The method of claim 12, wherein administering the composition comprises administering a composition further including an herb in an amount that increases endothelial nitric oxide synthetase activity in the body of the subject.

16. The method of claim 15, wherein administering the composition comprises administer a composition in which the herb comprises ginkgo biloba.

17. The method of claim 12, wherein administering the composition comprises administering a composition further including a food product in an amount that causes the body of the subject to increase nitric oxide production.
